(19) United States
(12) Patent Application Publication
(10) Pub. No.: US 2022/0211411 A1
(43) Pub. Date: Jul. 7, 2022

(54) NEEDLE ASSEMBLY

(71) Applicant: Michael Forney, Pepper Pike, OH (US)

(72) Inventor: Michael Forney, Pepper Pike, OH (US)

(21) Appl. No.: 17/142,091

(22) Filed: Jan. 5, 2021

(51) Int. Cl. A61B 17/34

(52) U.S. Cl. CPC A61B 17/3403; A61B 2017/3413; A61B 17/3423

(57) ABSTRACT

A needle assembly comprises an outer cannula having a longitudinally extending cannula wall with a sharpened distal tip. An inner surface of the cannula wall defines a longitudinally extending cannula lumen. A plunger housing is connected to a proximal end portion of the outer cannula. A plunger is received in the plunger housing and is spring biased in a proximal direction. An inner surface of a longitudinally extending plunger wall defines a plunger lumen aligned with the cannula lumen. The inner surface of a proximal end portion of the plunger wall has an orientation specific configuration. A needle is receivable in the plunger and cannula lumens. An outer surface of a proximal end portion of a longitudinally extending needle wall has an orientation specific configuration complementary to and fitting within the orientation specific configuration of the plunger wall. A cap covers the distal end portions of the plunger and the needle when the needle is received in the plunger and cannula lumens.

### TECHNICAL FIELD

The present disclosure relates to a needle assembly for ultrasound guided fenestration and/or transection of tissue and, more particularly, to a needle assembly in which a plunger-operated needle has a specific orientation with respect to an outer cannula.

### BACKGROUND

Ultrasound guided musculoskeletal interventions are rapidly becoming more common and often offer an alternative to more costly and risky surgeries. Ultrasound guided fenestration of ligaments and tendons using a needle alone has been performed. Such needle fenestration, however, does not appear to offer a robust equivalent to endoscopic or open techniques because needling does not transect tissue in the manner a surgeon does with a scalpel.

### SUMMARY

The present disclosure relates to a needle assembly for ultrasound guided fenestration and/or transection of tissue and, more particularly, to a needle assembly in which a plunger-operated needle has a specific orientation with respect to an outer cannula.

In certain aspects, a needle assembly is provided that includes an outer cannula having a longitudinally extending cannula wall with an inner surface and an outer surface. The inner surface of the cannula wall defines a cannula lumen extending longitudinally through the outer cannula. The cannula wall includes a distal end portion and a proximal end portion. The distal end portion of the cannula wall includes a sharpened tip. The needle assembly also comprises a plunger housing connected to the proximal end portion of the outer cannula and a plunger received in the plunger housing. The plunger has a longitudinally extending plunger wall with an inner surface and an outer surface. The inner surface of the plunger wall defines a plunger lumen extending longitudinally through the plunger. The plunger lumen is axially aligned with the cannula lumen. A proximal end portion of the plunger protrudes from the plunger housing. The inner surface of the plunger wall within the proximal end portion of the plunger has an orientation specific configuration. The needle assembly further comprises a spring received in the plunger housing and biasing the plunger in a proximal direction. A needle is receivable axially in the plunger lumen and in the cannula lumen. The needle has a longitudinally extending needle wall with an outer surface. A proximal end portion of the needle is receivable in the proximal end portion of the plunger. The outer surface of the needle wall in the proximal end portion of the needle has an orientation specific configuration that is complementary to and fits within the orientation specific configuration of the inner surface of the plunger wall. A cap is configured to cover the proximal end portion of the plunger and the proximal end portion of the needle when the needle is received in the plunger lumen and in the cannula lumen.

In certain aspects, a needle assembly is provided that comprises an outer cannula having a distal end including a sharpened tip, a proximal end, and an outer cannula lumen extending longitudinally therebetween. A plunger housing is connected to the proximal end of the outer cannula. A plunger is received in the plunger housing, the plunger having a distal portion, a proximal portion, and a plunger lumen extending longitudinally therebetween and being axially aligned with the cannula lumen. The proximal portion has a proximal face defining an orientation-specific opening. A spring is received in the plunger housing and biases the plunger in a proximal direction. A needle is receivable axially in the plunger lumen and in the cannula lumen. The needle has a distal portion, a proximal portion, and a needle lumen extending therebetween. The proximal portion has a proximal end key having an orientation-specific configuration complimentary to and releasably lockable into the orientation-specific opening in the proximal portion of the plunger. The needle assembly further includes an end cap configured to cover the proximal portion of the plunger and the proximal portion of the needle when the needle is received in the plunger lumen and in the cannula lumen. The needle assembly further comprises a handle extending laterally from the plunger housing and having a fixed orientation when the orientation-specific proximal end key of the needle locks into the complimentary orientation specific opening at the proximal portion of the plunger.

### BRIEF DESCRIPTION OF THE DRAWINGS

FIG. 1 is a side view, partly in section, of a needle assembly in accordance with an embodiment of the present disclosure;

FIG. 2 is a side view, partly in section, of the needle assembly of FIG. 1 with certain components in different positions;

FIG. 3 is a perspective view, partly exploded, of certain components of the needle assembly of FIG. 1;

FIG. 4 is another perspective view of certain components of the needle assembly of FIG. 1;

FIG. 5 is still another perspective view of certain components of the needle assembly of FIG. 1;

FIGS. 6A, 6B, and 6C are schematic views of different cutting needle tips usable in the needle assembly of FIG. 1; and

FIG. 7 is side view, partly in section, of a needle assembly in accordance with another embodiment of the present disclosure.

### DETAILED DESCRIPTION

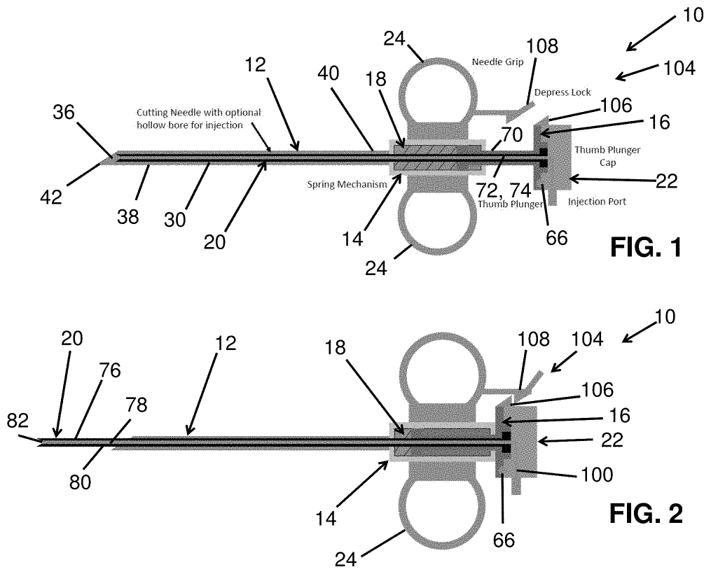

FIGS. 1 through 5 illustrate a needle assembly 10 for ultrasound guided fenestration and/or transection of tissue, in accordance with an example of the present invention. The needle assembly 10 comprises an outer cannula 12, a plunger housing 14, a plunger 16, a spring 18, a cutting needle 20, an end cap 22, and a handle, which in the illustrated examples, is two finger grips 24.

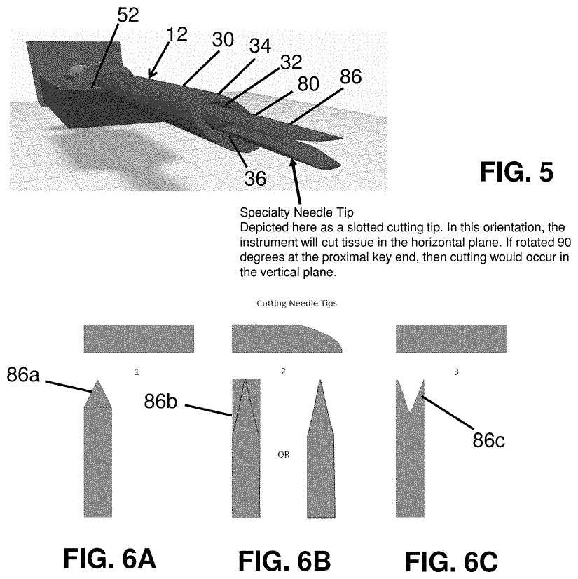

The outer cannula 12 includes a longitudinally extending cannula wall 30 that can be circular in cross-section. As best seen in FIG. 5, the cannula wall 30 has an inner surface 32 and an outer surface 34. The inner surface 32 of the cannula wall 30 defines a cannula lumen 36 that extends longitudinally through the outer cannula 12. The cannula wall 30, as shown in FIG. 1, also includes a distal end portion 38 and a proximal end portion 40. The distal end portion 38 of the cannula wall 30 is sharpened such that the cannula wall and the outer cannula 12 have a sharpened and pointed tip 42. The proximal end portion 40 of the cannula wall 30 is fixed, secured, or permanently attached to the plunger housing 14.

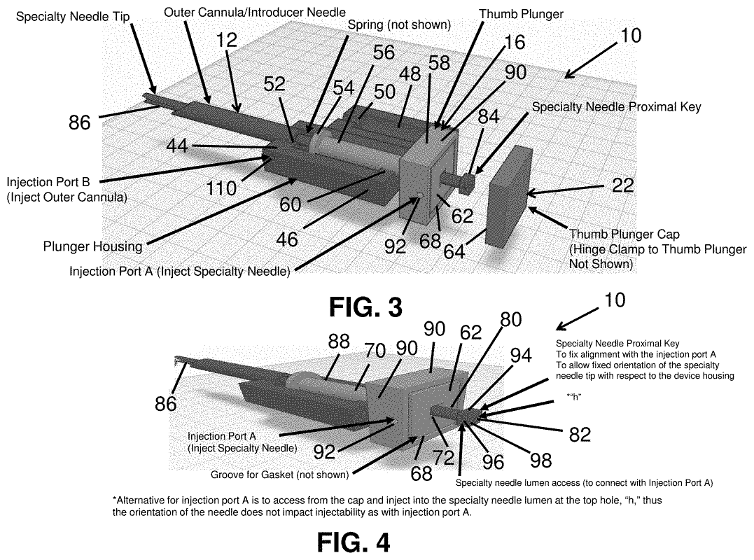

As best seen in FIG. 3, the plunger housing 14 can have a rectangular outer shape. The plunger housing 14 may be formed in two halves, as shown, to facilitate assembly of the plunger housing with the outer cannula 12 and the plunger 16. The plunger housing 14 has a distal end portion 44 and a proximal end portion 46. The plunger housing 14 also can have a cylindrical inner surface 48 that extends throughout the length of the plunger housing and that defines a housing bore 50. The housing bore 50 extends through the entire length of the plunger housing 14 but can have different diameters along its length. In the distal end portion 44 of the plunger housing 14, the housing bore 50 has a diameter that is the same as or slightly larger than the outer diameter of the cannula wall 30 of the outer cannula 12. The proximal end portion 40 of the outer cannula 12 may thus be received in the housing bore 50 with a friction fit against the inner surface 48 of the plunger housing 14 so as to be fixed, secured, or permanently attached to the plunger housing. In addition or alternatively, the proximal end portion 40 of the outer cannula 12 may be fixed, secured, or permanently attached to the plunger housing 14 with adhesive or welding or any other suitable attachment technique. To help ensure that the proximal end portion 40 of the outer cannula 12 is properly positioned relative to the plunger housing 14, the housing bore 50 in the distal end portion 44 of the plunger housing may have a second, smaller diameter to provide an abutment or wall 52 against which the proximal end of the outer cannula may abut.

Proximal of the wall 52, the housing bore 50 increases in diameter to receive the plunger 16. The plunger 16 includes a distal end portion 54, an intermediate portion 56, and a proximal end portion 58. The distal end portion 54 and the intermediate portion 56 both can have cylindrical outer surfaces. The outer diameter of the distal end portion 54 is greater than the outer diameter of the intermediate portion 56 so that the distal end portion effectively provides a retaining flange. The retaining flange defined by the distal end portion 54 of the plunger 16 engages and retains one end of the spring 18, which is shown schematically in FIGS. 1 and 2. The spring 18 is received in the housing bore 50 and may, for example, be a coil spring with an inner cylindrical space such that the spring may encircle the cutting needle 20. The opposite end of the spring 18 is engaged by and retained by the wall 52. The spring 18 thus tends to push or bias the distal end portion 54 of the plunger 16 away from the wall 52 or in a proximal direction.

To accommodate or allow a range of travel for the plunger 16, the housing bore 50, for a majority of its length, can have a diameter that is the same as or slightly larger than the diameter of the distal end portion 54 of the plunger. In the proximal end portion 46 of the plunger housing 14, particularly at the proximal end of the plunger housing, the housing bore 50 can have a diameter that is the same as or slightly larger than the diameter of the intermediate portion 56 of the plunger 16. The proximal end portion 46 of the plunger housing 14 thus effectively provides a retaining wall 60 to engage the distal end portion 54 of the plunger 16 and thereby limit travel of the plunger 16 in a proximal direction and help retain the plunger in the plunger housing.

The proximal end portion 58 of the plunger 16 can be located and always remain outside of the plunger housing 14. The proximal end portion 58 also can have an outer periphery that is larger than the outer periphery of the intermediate portion 56 of the plunger 16. The proximal end portion 58 of the plunger 16 thus limits the throw of the plunger 16 or, stated differently, the distance through which the plunger can travel in a proximal direction relative to the plunger housing 14 and the outer cannula 12. In addition to the throw limitation provided by the proximal end portion 58 of the plunger 16, further limits on the throw of the plunger may be provided by an optional adjustable washer (not shown) disposed around the outer surface of the intermediate portion 56 of the plunger between the retaining wall 60 of the proximal end portion 46 of the plunger housing 14 and the proximal end portion of the plunger. Such an adjustable washer would limit the proximal travel of the plunger 16 by contacting the retaining wall 60 before the proximal end portion 58 of the plunger. Such an adjustable washer would be movable axially to different positions along the outer surface of the intermediate portion 56 of the plunger 16 and fixed in placed in a desired position using a set screw or other locking mechanism. As shown, the proximal end portion 58 of the plunger 16 has an outer periphery or outer shape that is square. The proximal end portion 58 may, however, have a circular outer shape or any other convenient outer shape so long as the outer periphery of the proximal end portion is greater than the outer periphery of the intermediate portion 56.

The proximal end portion 58 of the plunger 16 has a proximal end face 62, which can engage a distal end face 64 of the end cap 22. The end cap 22 is attached to the proximal end portion 58 of the plunger 16 via a hinge 66, as shown schematically in FIGS. 1 and 2. The end cap 22 may thus be pivoted in a first direction around the hinge 66 away from the proximal end portion 58 of the plunger 16 so that the proximal end face 62 of the proximal end portion is exposed. The end cap 22 may also be pivoted in a second, opposite direction around the hinge 66 toward the proximal end portion 58 of the plunger 16 so that the proximal end face 62 of the proximal end portion is covered by the end cap. To retain the cap 22 in a position covering the proximal end face 62 of the proximal end portion 58 of the plunger 16, the distal end face 64 of the end cap and the proximal end face of the proximal end portion 58 of the plunger 16 may have complementary configurations so that the end cap is held against the proximal end portion of the plunger via a friction fit or a snap fit. Alternatively, a latch mechanism (not shown) may have one portion attached to the end cap 22 and a second portion attached to the proximal end portion 58 of the plunger 16 so that the first and second portions of the latch mechanism may be engaged with one another to hold the distal end face 64 of the end cap against the proximal end face 62 of the proximal end portion of the plunger. Also, one or both of the distal end face 64 of the end cap 22 and the proximal end face 62 of the proximal end portion 58 of the plunger 16 may be formed with a groove 68 to receive a gasket (not shown), which provides a fluid seal between the cap and the proximal end portion of the plunger.

The plunger 16 has a longitudinally extending plunger wall 70. The plunger wall has an inner surface 72 that extends throughout the length of the plunger. The inner surface 72 defines a plunger lumen 74 that also extends through the entire length of the plunger 16. Throughout a majority of its length, the inner surface 72 can have a circular cross-section and, thus, the plunger lumen 74 can have a corresponding circular cross-section. Adjacent the proximal end face 62 of the proximal end portion 58 of the plunger 16, however, the inner surface 72 has a different, orientation-specific cross-section or configuration. In particular, the proximal end face can define an orientation-specific opening. As shown in FIGS. 3 and 4, for example, the orientation-specific opening is square, but other configurations may be used, such as triangular or star-shaped. Suitable orientation-specific openings are selected in conjunction with the shape of the cutting needle 20. Specifically, the cutting needle 20 is received in the plunger lumen 74. The cutting needle 20 has a longitudinally extending needle wall 76. The needle wall 76 has an inner surface 78 and an outer surface 80. The inner surface 78 defines a needle lumen 82 that extends longitudinally throughout the entire length of the cutting needle 20. Throughout its length, the inner surface 78 can have a circular cross-section and, thus, the needle lumen 82 can have a corresponding circular cross-section. For a majority of its length, the outer surface 80 also can have a circular cross-section. In a proximal end portion 84 of the cutting needle 20, however, the outer surface 80 has a different, orientation-specific cross-section or configuration. In particular, the proximal portion of the needle can have a proximal end key having an orientation-specific configuration complimentary to and releasably lockable into the orientation specific opening in the proximal portion of the plunger. As shown in FIGS. 3 and 4, for example, the orientation-specific proximal end key is square, but other configurations may be used.

The orientation-specific configuration of the outer surface 80 of the needle wall 76 and thus the cutting needle 20 is complementary to and fits within the orientation-specific configuration of the inner surface 72 of the plunger wall 70 adjacent the proximal end face 62 of the proximal end portion 58 of the plunger 16. The complementary orientation-specific configurations limit the number of orientations of the cutting needle 20 relative to the plunger 16 and to a longitudinal central axis of the plunger. With a square configuration, the number of possible relative orientations is limited to four. If the configuration were a triangle, the number of possible relative orientations would be limited to three. Other orientation-specific configurations might limit the number of possible relative orientations to two or one or to a number greater than four.

The complementary orientation-specific configurations of (a) the outer surface 80 of the needle wall 76 in the proximal end portion 84 of the cutting needle 20 and (b) the inner surface 72 of the plunger wall 70 adjacent the proximal end face 62 of the proximal end portion 58 of the plunger 16 facilitate the delivery of fluid, such as a liquid anesthetic, to the needle lumen 82 and also help to ensure a known orientation among the outer cannula 12, including its sharpened and pointed tip 42, a distal end portion 86 of the cutting needle, and the handle (e.g. finger grips 24). More particularly, the plunger wall 70 has an outer surface 88 that defines the outer periphery or outer shape of the plunger 16. In the proximal end portion 58 of the plunger 16, the outer surface 88 of the plunger wall 70 can have a square configuration with four sides 90. At least one of the sides 90 is provided with a plunger port 92, which is a bore or passageway that extends from the outer surface 88 of the plunger wall 70 to the inner surface 72. The plunger port 92 thus permits the delivery of fluid from outside of the plunger 16 to the plunger lumen 74.

Similarly, in the proximal end portion 84 of the cutting needle 20, the outer surface 80 of the needle wall 76 also can have a square configuration with four sides 94. At least one of the sides 90 can be provided with a needle port 96, which is a bore or passageway that extends from the outer surface 80 of the needle wall 76 to the inner surface 78 of the needle wall. The needle port 96 thus permits the delivery of fluid from outside of the cutting needle 20 to the needle lumen 82. The complementary orientation-specific configurations of (a) the outer surface 80 of the needle wall 76 in the proximal end portion 84 of the cutting needle 20 and (b) the inner surface 72 of the plunger wall 70 adjacent the proximal end face 62 of the proximal end portion 58 of the plunger 16 help to ensure that the plunger port 92 can be readily aligned with the needle port 96 to permit the delivery of fluid, such as a liquid anesthetic, to the needle lumen 82.

In addition to or as an alternative to communicating with the needle port 96, the needle lumen 82 may extend into a distal end face 98 of the proximal end portion 84 of the cutting needle 20. By providing an appropriate cap port 100, which is a bore or passageway that extends from an outer surface 102 of the end cap 22 to the distal end face 64 of the end cap, fluid may be delivered from outside of the end cap to the proximal end of the needle lumen 82. Such a cap port 100 permits the delivery of fluid to the needle lumen regardless of the orientation of the cutting needle 20 relative to the plunger 16, but adds some complexity to the design of the end cap 22.

As mentioned above, the complementary orientation-specific configurations of (a) the outer surface 80 of the needle wall 76 in the proximal end portion 84 of the cutting needle 20 and (b) the inner surface 72 of the plunger wall 70 adjacent the proximal end face 62 of the proximal end portion 58 of the plunger 16 also help to ensure a known orientation among the outer cannula 12, including its sharpened and pointed tip 42, the distal end portion 86 of the cutting needle, and the handle such as finger grips 24. More particularly, the distal end portion 86 of the cutting needle 20 may have a configuration, such as the slotted cutting tip shown in FIG. 5, that makes it important for the surgeon or other healthcare professional using the needle assembly 10 to be able confidently to know the orientation of the distal end portion without being able to see the distal end portion directly (i.e., without the aid of ultrasound, x-ray, or other imaging technology).

To facilitate grasping and manipulating the needle assembly 10, the handle such as finger grips 24 are fixed, secured, or permanently attached to the plunger housing 14 in a desired orientation, such as, on opposites sides of the plunger housing. Because the outer cannula 12 is also fixed, secured, or permanently attached to the plunger housing 14, the handle, such as finger grips 24, are disposed in fixed or known orientation relative to the outer cannula, including its sharpened and pointed tip 42. By appropriately arranging the complementary orientation-specific configurations of the outer surface 80 of the needle wall 76 in the proximal end portion 84 of the cutting needle 20 and the inner surface 72 of the plunger wall 70 adjacent the proximal end face 62 of the proximal end portion 58 of the plunger 16, the handle, such as finger grips 24, will also be disposed in fixed or known orientation relative to the cutting needle, including its specially configured distal end portion 86. When the surgeon or other healthcare professional using the needle assembly 10 grasps the needle assembly by placing two fingers in the finger grips 24, the surgeon or other healthcare professional will be able to establish the relative orientations of his or her hand grasping the needle assembly, the outer cannula, including its sharpened and pointed tip 42, and the cutting needle 20, including its specially configured distal end portion 86. The surgeon or other healthcare professional using the needle assembly 10 will then be able confidently and reliably to manipulate the needle assembly to treat a patient.

The needle assembly 10 will typically be assembled, for example, at a manufacturing facility, such that the finger grips 24 and the proximal end portion 40 of the outer cannula 12 are fixed, secured, or permanently attached to the plunger housing 14, the distal end portion 54 and the intermediate portion 56 of the plunger 16 are received in the plunger housing, and the spring 18 is also received in the plunger housing between the wall 52 and the distal end portion of the plunger. To prepare the needle assembly 10 for a particular procedure, a surgeon or other healthcare provider will select a particular cutting needle 20 with a desired configuration of its distal end portion 86 and move or pivot the end cap 22 around the hinge 66 away from the proximal end face 62 of the proximal end portion 58 of the plunger 16 to expose the open proximal end of the plunger lumen 74. The surgeon or other healthcare provider will insert the distal end portion 86 of the cutting needle 20 lengthwise or longitudinally into the plunger lumen 74 and push the cutting needle through the central opening of the spring 18 and into the cannula lumen 36 of the outer cannula 12. The surgeon or other healthcare provider will then manipulate the cutting needle 20 so that the orientation-specific configuration of the outer surface 80 of the needle wall 76 and, thus, the cutting needle fits within the orientation-specific configuration of the inner surface 72 of the plunger wall 70 adjacent the proximal end face 62 of the proximal end portion 58 of the plunger 16. By pressing the cutting needle 20 until it seats within the orientation-specific configuration of the inner surface 72 of the plunger wall 70 adjacent the proximal end face 62 of the proximal end portion 58 of the plunger 16, the surgeon or other healthcare provider will have established the relative orientations of the finger grips 24, the outer cannula 12, including its sharpened and pointed tip 42, and the cutting needle, including its specially configured distal end portion 86. To hold the cutting needle 20 in position, the cap 22 will be moved or pivoted around the hinge 66 until the distal end face 64 of the cap engages and locks into position relative to the proximal end face 62 of the proximal end portion 58 of the plunger 16.

In use, after the assembly procedure set out above, the finger grips 24 of the needle assembly 10 will be grasped by the surgeon or other healthcare provider. If a procedure to be performed by the surgeon or other healthcare provider includes injecting a fluid, such as a liquid anesthetic, into a patient's tissue, a source of the fluid (not shown) will be connected in fluid communication with the plunger port 92 and/or the cap port 100. The surgeon or other healthcare provider will then insert the sharpened and pointed tip 42 of the outer cannula 12 into the patient's tissue. When the outer cannula 12 is appropriately positioned in the patient's tissue, the surgeon or other healthcare provider may deploy the distal end portion 86 of the cutting needle 20 by pressing on the end cap 22 with his or her thumb to move the cap, the plunger 16 and the cutting needle in a distal direction so that the distal end portion of the cutting needle moves into the patient's tissue. The surgeon or other healthcare provider may then allow the distal end portion 86 of the cutting needle 20 to retract or move in a proximal direction into the outer cannula 12 under the bias of the spring 18. The surgeon or other healthcare provider may repeat the process of distal extension and proximal retraction of the cutting needle 20 on an intermittent basis while manipulating the outer cannula 12 to adjust the position of the sharpened and pointed tip 42 of the outer cannula in the patient's tissue.

Although the handle is illustrated in the figures as opposing finger grips 24 having ring shapes to accommodate a user's fingers, the handle can have other configurations as well, such as a trigger shape that is pressed or pulled to actuate components of the needle assembly.

Once the cutting needle 20 is fully deployed or extended longitudinally relative to the outer cannula 12 to the full extent of the available travel of the plunger 16 in the plunger housing 14, the surgeon or other healthcare provider may desire to keep the cutting needle fully deployed without applying continuous pressure on the cap 22. To permit keeping the cutting needle 20 fully deployed without applying continuous pressure on the cap 22, the needle assembly 10 may be provided with an optional lock assembly 104. As shown in FIGS. 1 and 2, the optional lock assembly 104 includes a first latch portion 106 attached to the proximal end portion 58 of the plunger 16 and a second latch portion 108 attached to one of the finger grips 24. Interference engagement between the first and second latch portions 106 and 108 holds the lock assembly 104 in its latched condition and keeps the cutting needle 20 fully deployed. Although a particular lock assembly 104 is shown, other lock assemblies may be used.

While the foregoing description of a procedure in which a surgeon or other healthcare provider uses the needle assembly 10 involves the use of a cutting needle 20 having a particular configuration for its distal end portion 86, the surgeon or other healthcare provider may wish to employ a plurality of cutting needles 20 having different configurations for their respective distal end portions 86. Because the cutting needle 20 is installed in the needle assembly 10 by inserting the cutting needle lengthwise or longitudinally into the plunger lumen 74 and the cannula lumen 36, the cutting needle may be removed from the needle assembly by extracting the cutting needle lengthwise or longitudinally from the cannula lumen and the plunger lumen. A particular cutting needle 20 may thus be removed from the needle assembly 10 and replaced with a different cutting needle by a surgeon or other healthcare provider during a procedure without removing the needle assembly from a patient's tissue or adjusting the position of the sharpened and pointed tip 42 of the outer cannula 12. The surgeon or other healthcare provider may thereby use different cutting needles 20 having different properties and capabilities, such different distal end portions 86 or different surface roughnesses, during a single surgical procedure.

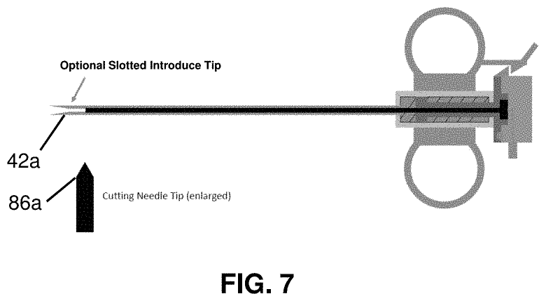

FIG. 5 illustrates a particular configuration for the distal end portion 86 of the cutting needle 20. Other configurations may be more appropriate for various procedures, however. Three such other configurations and other distal end portions 86a, 86b, and 86c are shown in FIGS. 6A, 6B, and 6C. In each of FIGS. 6A, 6B, and 6C, a side view of each distal end portion 86a, 86b or 86c is shown above a view taken from a position ninety (90°) degrees away from the side view. Other configurations may also be provided for the sharpened and pointed tip 42 of the outer cannula 12. One such other configuration and sharpened and pointed tip 42a is shown in FIG. 7, together with a different configuration for the distal end portion 86a of the cutting needle 20. The cutting needle 20 may optionally not have an inner surface 78 or a needle lumen 82. Such an optional solid needle would still have a distal end portion suitable for fenestration and/or transection, but would not be capable of delivering a fluid to a patient's tissue.

While the needle assembly 10 has been described as being configured to deliver a fluid to and through the needle lumen 82, it may also be desirable to deliver fluid to or extract fluid from the cannula lumen 36. To enable such delivery or extraction of fluid, a cannula port 110, which is a bore or passageway that extends from the outer surface 34 of the outer cannula 12 to the inner surface 32 of the outer cannula, may be provided in the outer cannula. Such a cannula port 110 permits the delivery of fluid from outside the outer cannula 12 to the cannula lumen 36 and/or extraction of fluid from the cannula lumen to the outside of the outer cannula.

In addition, while the cutting needle 20 and the outer cannula 12 have been shown as being straight along their respective lengths, they may also be curved in a longitudinal direction. If the outer cannula 12 is curved, the cutting needle 20 may either have a complementary curved configuration along its length or be sufficiently flexible to accommodate being received in a curved outer cannula. Further, for certain procedures, the cutting needle 20 may not perform any cutting, but may simply be used for fenestration of a patient's tissue. In such a situation, the cutting needle 20 may simply have a pointed tip, like a typical injection needle. Further still, it may be desirable initially to position the outer cannula 12 using a solid stylet, which would be removed and replaced with a cutting needle 20 or fenestration needle after the outer cannula is appropriately positioned.

From the above description of the invention, those skilled in the art will perceive improvements, changes and modifications. Such improvements, changes, and/or modifications within the skill of the art are intended to be covered by the appended claims. For example, while the above described assembly has been disclosed with respect to a needle assembly, another instrument could be used with components of the assembly. For example, an instrument could be inserted into the cannula lumen of the the outer cannula and lock into the plunger proximal portion. Such an instrument could be used to cut tissue but also could be used to fenestrate tissue or be blunt to dissect or manipulate tissue. Also this instrument may or may not have an inner lumen itself through which fluid could be injected via a side port of the plunger. As such, the present disclosure covers various assemblies with an ultrasound guided surgical port through which a variety of instruments could be inserted in a locked orientation into an outer cannula.

What is claimed is:

1. An instrument assembly comprising:
an outer cannula having a distal end, a proximal end, and a cannula lumen extending longitudinally therebetween;
a plunger housing connected to the proximal end of the outer cannula;
a plunger received in the plunger housing, the plunger having a distal portion, a proximal portion, and a plunger lumen extending longitudinally therebetween and being axially aligned with the cannula lumen, the proximal portion having a proximal face defining an orientation-specific opening;
a spring received in the plunger housing and biasing the plunger in a proximal direction;
an instrument receivable axially in the plunger lumen and in the cannula lumen, the instrument having a distal portion, and a proximal portion, the proximal portion having an proximal end key having an orientation-specific configuration complimentary to and releasably lockable into the orientation-specific opening in the proximal portion of the plunger;
an end cap configured to cover the proximal portion of the plunger and the proximal portion of the instrument when the instrument is received in the plunger lumen and in the cannula lumen; and
a handle extending laterally from the plunger housing and having a fixed orientation when the orientation-specific proximal end key of the instrument locks into the complimentary orientation specific opening at the proximal portion of the plunger.

2. The instrument assembly according to claim 1, wherein the instrument is a needle.

3. The instrument assembly according to claim 2, wherein:
the needle comprises a longitudinally extending needle wall with an inner surface and an outer surface, the inner surface defining the needle lumen; and
the plunger comprises a longitudinally extending plunger wall with an inner surface and an outer surface, the inner surface defining the plunger lumen.

4. The instrument assembly according to claim 3, wherein a needle injection port extends laterally through the needle wall in the proximal portion of the needle so as to communicate with the needle lumen, a plunger injection port extends laterally through the plunger wall in the proximal portion of the plunger so as to communicate with the needle injection port when the needle is received in the plunger lumen and in the cannula lumen.

5. The instrument assembly of claim 1, wherein the distal end of the outer cannula or the instrument includes a sharpened tip.

6. The instrument assembly of claim 1, wherein the distal end of the outer cannula or the instrument includes a blunt tip.

7. The instrument assembly according to claim 1, wherein:
the instrument comprises a longitudinally extending instrument wall with an inner surface and an outer surface;
the plunger comprises a longitudinally extending plunger wall with an inner surface and an outer surface, the inner surface defining the plunger lumen.

8. The instrument assembly according to claim 7, wherein at least a portion of the inner surface of the plunger wall in the proximal portion of the plunger is enlarged in at least one direction transverse to the longitudinal extent of the plunger lumen, at least a portion of the outer surface of the instrument wall in the proximal portion of the instrument being correspondingly enlarged in at least one direction transverse to the longitudinal extent of the instrument, the proximal portion of the instrument being captured between the end cap and the at least a portion of the inner surface of the plunger wall in the proximal portion of the plunger when the instrument is received in the plunger lumen and in the cannula lumen and the end cap is covering the proximal portion of the plunger and the proximal portion of the instrument.

9. A needle assembly according to claim 7, wherein an orientation of the outer surface of the instrument wall in the proximal portion of the instrument about a central longitudinal axis of the plunger is fixed when the instrument is received in the plunger lumen and in the cannula lumen and the outer surface of the instrument wall in the distal portion of the instrument is received within the inner surface of the plunger wall in the distal portion of the plunger.

10. The instrument assembly of claim 1, wherein the end cap has a distal face complimentary and releasably lockable to the proximal face of the plunger when the instrument is received in the plunger lumen and in the cannula lumen.
